SEQUENCE LISTING (8 sequences)

SEQ ID NO: 1 - protein, 121 aa, Artificial Sequence [sequence omitted]

SEQ ID NO: 2 - protein, 107 aa, Artificial Sequence [sequence omitted]

SEQ ID NO: 3 - protein, 451 aa, Artificial Sequence
  synthetic peptide
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Artificial Sequence
  synthetic peptide
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 120 aa, Artificial Sequence [sequence omitted]

SEQ ID NO: 6 - protein, 111 aa, Artificial Sequence [sequence omitted]

SEQ ID NO: 7 - protein, 447 aa, Artificial Sequence
  synthetic peptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 218 aa, Artificial Sequence
  synthetic peptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0233523 A1
(43) Pub. Date: Aug. 1, 2019

(54) COMBINATION THERAPY FOR CANCER

(71) Applicants: IMCLONE LLC, Indianapolis, IN (US); MERCK SHARP & DOHME CORP., Rahway, NJ (US)

(72) Inventors: Maciej Wojciech GIL, Katowice (PL); Raffael KUREK, Pforzheim (GE)

(21) Appl. No.: 16/330,128

(22) PCT Filed: Sep. 7, 2017
(86) PCT No.: PCT/US2017/050449

§ 371 Date: Mar. 4, 2019

Related U.S. Application Data
(60) Provisional application No. 62/399,715, filed on Sep. 26, 2016.

(51) Int. Cl. C07K 16/28; A61P 35/00; C07K 16/30

(52) U.S. Cl. CPC C07K 16/2863; A61P 35/00; C07K 16/2818; C07K 2317/515; C07K 2317/569; C07K 2317/565; C07K 2317/51; C07K 16/3023

(57) ABSTRACT

The present invention provides preparation of medicaments for use in treating and methods of treating cancer, in particular, non-small cell lung cancer, comprising an anti-EGFR antibody, preferably necitumumab, in combination with an anti-PD-1 antibody, preferably pembrolizumab.

The present invention relates to the field of medicine. The present invention relates to combinations of an antibody against human epidermal growth factor receptor (EGFR) and an antibody against human programmed cell death 1 (PD-1) receptor, for example the combination of necitumumab with pembrolizumab, and to methods of using the combinations to treat cancer, particularly lung cancer, more particularly, non-small cell lung cancer (NSCLC) specifically, squamous and nonsquamous.

Overexpression of EGFR has been reported in numerous human malignant conditions, including cancers of the bladder, brain, head and neck, pancreas, lung, breast, ovary, colon, prostate, and kidney. In many of these conditions, the overexpression of EGFR correlates or is associated with poor prognosis of the patients. Inhibition of EGFR signaling pathway has been used for treating cancer.

Necitumumab (IMC-11F8) is a recombinant human monoclonal antibody that binds to the extracellular domain III of the human EGFR and blocks interaction between EGFR and its ligands. Necitumumab, including sequences thereof, and methods of making and using this antibody, including for the treatment of neoplastic diseases such as solid and non-solid tumors, are disclosed in WO2005/090407.

Necitumumab is the active pharmaceutical ingredient in PORTRAZZA®, which has been approved for treating metastatic squamous NSCLC in combination with gemcitabine and cisplatin. The INSPIRE trial of pemetrexed and cisplatin with or without necitumumab, which enrolled NSCLC patients with nonsquamous histology, failed to meet its primary endpoint of overall survival. See the US and European labels of Portrazza® (necitumumab) (“Limitation of Use” and “Warnings and Precautions” section respectively).

The immune system is regulated by many inhibitory pathways, collectively referred to as immune checkpoints, which maintain the tolerance to self-antigens. One of the mechanisms of cancer resistance is the ability of tumor cells to co-opt immune checkpoint pathways. The adaptive upregulation of PD-L1, a ligand of PD-1, reflects the natural physiologic process of normal cell protection from immune-mediated tissue damage. This process, which is the most common in solid tumors, including NSCLC, is used by the cancer cells to avoid killing by the immune system.

Binding of the PD-1 ligands, PD-L1 and PD-L2, to the PD-1 receptor expressed on T cells, inhibits T cell proliferation and cytokine production. Upregulation of PD-1 ligands occur in some tumors and signaling through this pathway contributes to inhibition of active T-cell immune surveillance of tumors.

Pembrolizumab is a monoclonal antibody that binds to the human PD-1 receptor and blocks its interaction with PD-L1 and PD-L2, thereby releasing PD-1 pathway-mediated inhibition of the immune response, including the anti-tumor immune response. Pembrolizumab, sequences thereof, and methods of making and using this antibody, including for increasing the activity of an immune response through the PD-1 pathway, are disclosed WO 2008/156712.

Pembrolizumab is the active pharmaceutical ingredient in KEYTRUDA®, which has been approved by the FDA for the treatment of melanoma, NSCLC in a PD-L1 positive patient population, and squamous cell carcinoma of the head and neck. More specifically with regard to NSCLC, Keytruda® (pembrolizumab) is approved in the US for “patients with metastatic NSCLC whose tumors express PD-L1 as determined by an FDA-approved test and who have disease progression on or after platinum-containing chemotherapy” (“Indications of Use”). The approved indication for IHC PD-L1 IHC 22C3 pharmDx, Keytruda®'s FDA-approved PD-L1 test, provides that “[t]he specimen should be considered PD-L1 positive if TPS≥50% of the viable tumor cells exhibit membrane staining at any intensity.”

Lung cancer ranks as one of the most common causes of death due to cancer in both men and women throughout the world. The two main types of lung cancer are small cell lung cancer and NSCLC. NSCLC makes up approximately 85% or more of lung cancer cases. Treatment can involve surgery, chemotherapy, radiation therapy, biologic therapy, targeted therapy, and immunotherapy, as well as selected combinations of these treatments. Immunotherapy has recently emerged as a major therapeutic modality in several solid tumors, including NSCLC, but efficacy of specific combinations for these agents, dosing, sequencing, biomarkers/biomarker levels, mutations, and efficacy in varying disease states remains unknown.

Combinations of a PD-1 inhibitor and an EGFR inhibitor have been discussed. WO2015/176033 discusses nivolumab plus an EGFR-targeted tyrosine kinase inhibitor (TKI), including erlotinib, for the treatment of NSCLC. Rizvi N., J Clin Oncol 32:5s, 2014 (suppl; abstr 8022) discusses the results of NCT01454102, which studied “Safety and response with nivolumab (anti-PD-1; BMS-936558, ONO-4538) plus erlotinib in patients (pts) with epidermal growth factor receptor mutant (EGFR MT) advanced NSCLC.” NCT01454102 was performed with erlotinib, an EGFR TKI, in patients with mutations in EGFR. Bastos, B. et al. International Journal of Radiation Oncology, Biology, Physics, 2016; 94 (4), 929 discusses the use of nivolumab plus cetuximab for a proposed phase 1 clinical trial for treating recurrent head and neck cancer.

Unfortunately, a cure for NSCLC still remains elusive and even therapeutic options remain limited with poor prognosis for patients; treatments for NSCLC remain a continuing unmet clinical need. There exists a need for more and different therapies that may prove to be effective in treating NSCLC.

Novel methods for use of the combination of necitumumab and pembrolizumab to treat squamous and nonsquamous NSCLC are presented herein. Novel methods for use of the combination of Antibody 1 and Antibody 2 to treat squamous and nonsquamous NSCLC are presented herein. Accordingly, some aspects the present invention provide for Antibody 1 for use in simultaneous, separate, or sequential combination with Antibody 2 in the treatment of non-small cell lung cancer, wherein Antibody 1 comprises two heavy chains and two light chains, wherein the heavy chain comprises a heavy chain variable region (HCVR) having the amino acid sequence of SEQ ID NO: 1, and wherein the light chain comprises a light chain variable region (LCVR) having the amino acid sequence of SEQ ID NO: 2, and wherein Antibody 2 comprises two heavy chains and two light chains, wherein the heavy chain comprises a HCVR having the amino acid sequence of SEQ ID NO: 5, and the light chain comprises a LCVR having the amino acid sequence of SEQ ID NO: 6, and wherein Antibody 1 is administered at a dose of a dose of 400 mg to 800 mg, and wherein Antibody 2 is administered at a dose of 200 mg.

More particularly, Antibody 1 comprises two heavy chains and two light chains, comprising the heavy chain having the amino acid sequence of SEQ ID NO: 3, and the light chain having the amino acid sequence of SEQ ID NO: 4, and wherein Antibody 2 comprises two heavy chains and two light chains, comprising the heavy chain having the amino acid sequence of SEQ ID NO: 7, and the light chain having the amino acid sequence of SEQ ID NO: 8.

More particularly, Antibody 1 is administered at a dose of 400 mg. More particularly, Antibody 1 is administered at a dose of 600 mg. More particularly, Antibody 1 is administered at a dose of 800 mg.

In a preferred aspect, Antibody 1 is administered by intravenous infusion. In a preferred aspect, Antibody 2 is administered by intravenous infusion.

More particularly, the non-small cell lung cancer is nonsquamous. More particularly, the non-small cell lung cancer is squamous.

More particularly, Antibody 1 is administered at a dose of 400 mg on days 1 and 8 of each 3-week cycle and Antibody 2 is administered at a dose of 200 mg on day 1 of each 3-week cycle. More particularly, Antibody 1 is administered at a dose of 600 mg on days 1 and 8 of each 3-week cycle and Antibody 2 is administered at a dose of 200 mg on day 1 of each 3-week cycle. More particularly, Antibody 1 is administered at a dose of 800 mg on days 1 and 8 of each 3-week cycle and Antibody 2 is administered at a dose of 200 mg on day 1 of each 3-week cycle.

In a preferred aspect, Antibody 1 is administered by intravenous infusion. In a preferred aspect, Antibody 2 is administered by intravenous infusion.

More particularly, the non-small cell lung cancer is nonsquamous. More particularly, the non-small cell lung cancer is squamous.

More particularly, the present invention provides for Antibody 1 and Antibody 2 for use wherein a tumor tissue from the patient has a protein expression level of PD-L1, and wherein the expression of PD-L1 is less than 50% of the viable tumor cells showing partial or complete membrane staining.

More particularly, the present invention provides for Antibody 1 for use in simultaneous, separate or sequential combination with Antibody 2 in the treatment of non-small cell lung cancer in a patient, comprising in vitro assaying a sample of the patient's tumor tissue, for the protein expression level of PD-L1, and administering a therapeutically effective amount of Antibody 1 and Antibody 2, if the expression of PD-L1 is less than 50% of the viable tumor cells showing partial or complete membrane staining.

More particularly, the present invention provides for Antibody 1 for use in simultaneous, separate or sequential combination with Antibody 2 in the treatment of non-small cell lung cancer in a patient, comprising in vitro assaying a sample of the patient's tumor tissue, for the protein expression level of PD-L1, determining the level of PD-L1, and administering a therapeutically effective amount of Antibody 1 and Antibody 2, if the level of PD-L1 is less than 50% of the viable tumor cells showing partial or complete membrane staining.

The present invention also provides for Antibody 2 for use in simultaneous, separate, or sequential combination with Antibody 1 for the treatment of non-small cell lung cancer, wherein Antibody 1 comprises two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and wherein Antibody 2 comprises two heavy chains and two light chains, wherein the heavy chain comprises a HCVR having the amino acid sequence of SEQ ID NO: 5, and the light chain comprises a LCVR having the amino acid sequence of SEQ ID NO: 6, and wherein Antibody 2 is administered at a dose of 200 mg, and wherein Antibody 1 is administered at a dose of a dose of 400 mg to 800 mg.

More particularly, Antibody 1 comprises two heavy chains and two light chains, comprising the heavy chain having the amino acid sequence of SEQ ID NO: 3, and the light chain having the amino acid sequence of SEQ ID NO: 4, and wherein Antibody 2 comprises two heavy chains and two light chains, comprising the heavy chain having the amino acid sequence of SEQ ID NO: 7, and the light chain having the amino acid sequence of SEQ ID NO: 8.

More particularly, Antibody 1 is administered at a dose of 400 mg. More particularly, Antibody 1 is administered at a dose of 600 mg. More particularly, Antibody 1 is administered at a dose of 800 mg.

More particularly, Antibody 2 is administered at a dose of 200 mg on day 1 of each 3-week cycle and Antibody 1 is administered at a dose of 400 mg on days 1 and 8 of each 3-week cycle. More particularly, Antibody 2 is administered at a dose of 200 mg on day 1 of each 3-week cycle, and Antibody 1 is administered at a dose of 600 mg on days 1 and 8 of each 3-week cycle. More particularly, Antibody 2 is administered at a dose of 200 mg on day 1 of each 3-week cycle, and Antibody 1 is administered at a dose of 800 mg on days 1 and 8 of each 3-week cycle.

In a preferred aspect, Antibody 1 is administered by intravenous infusion. In a preferred aspect, Antibody 2 is administered by intravenous infusion.

More particularly, the non-small cell lung cancer is nonsquamous. More particularly, the non-small cell lung cancer is squamous.

More particularly, the present invention provides for Antibody 2 for use wherein a tumor tissue from the patient has a the protein expression level of PD-L1 if the expression of PD-L1 is less than 50% of the viable tumor cells showing partial or complete membrane staining.

More particularly, the present invention provides for Antibody 2 for use in simultaneous, separate or sequential combination with Antibody 1 in the treatment of non-small cell lung cancer in a patient, comprising in vitro assaying a sample of the patient's tumor tissue, for the protein expression level of PD-L1, and administering a therapeutically effective amount of Antibody 2 and Antibody 1, if the expression of PD-L1 is less than 50% of the viable tumor cells showing partial or complete membrane staining.

More particularly, the present invention provides for Antibody 2 for use in simultaneous, separate or sequential combination with Antibody 1 in the treatment of non-small cell lung cancer in a patient, comprising in vitro assaying a sample of the patient's tumor tissue, for the protein expression level of PD-L1, determining the level of PD-L1, and administering a therapeutically effective amount of Antibody 2 and Antibody 1, if the level of PD-L1 is less than 50% of the viable tumor cells showing partial or complete membrane staining.

The present invention also provides for use of Antibody 1 in the manufacture of a medicament for the treatment of non-small cell lung cancer, wherein Antibody 1 is administered at a dose of 400 mg to 800 mg in simultaneous, separate, or sequential combination with Antibody 2, wherein Antibody 2 is administered at a dose of 200 mg, and wherein Antibody 1 comprises two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and wherein Antibody 2 comprises two heavy chains and two light chains, wherein the heavy chain comprises a HCVR having the amino acid sequence of SEQ ID NO: 5, and the light chain comprises a LCVR having the amino acid sequence of SEQ ID NO: 6.

More particularly, Antibody 1 comprises two heavy chains and two light chains, comprising the heavy chain having the amino acid sequence of SEQ ID NO: 3, and the light chain having the amino acid sequence of SEQ ID NO: 4, and wherein Antibody 2 comprises two heavy chains and two light chains, comprising the heavy chain having the amino acid sequence of SEQ ID NO: 7, and the light chain having the amino acid sequence of SEQ ID NO: 8.

More particularly, Antibody 1 is administered at a dose of 400 mg. More particularly, Antibody 1 is administered at a dose of 600 mg. More particularly, Antibody 1 is administered at a dose of 800 mg.

More particularly, Antibody 1 is administered at a dose of 400 mg on days 1 and 8 of each 3-week cycle and Antibody 2 is administered at a dose of 200 mg on day 1 of each 3-week cycle. More particularly, Antibody 1 is administered at a dose of 600 mg on days 1 and 8 of each 3-week cycle and Antibody 2 is administered at a dose of 200 mg on day 1 of each 3-week cycle. More particularly, Antibody 1 is administered at a dose of 800 mg on days 1 and 8 of each 3-week cycle and Antibody 2 is administered at a dose of 200 mg on day 1 of each 3-week cycle.

In a preferred aspect, Antibody 1 is administered by intravenous infusion. In a preferred aspect, Antibody 2 is administered by intravenous infusion.

More particularly, the non-small cell lung cancer is nonsquamous. More particularly, the non-small cell lung cancer is squamous.

The present invention also provides for use of Antibody 2 in the manufacture of a medicament for the treatment of non-small cell lung cancer, wherein Antibody 2 is administered at a dose of 200 mg in simultaneous, separate, or sequential combination with Antibody 1, wherein Antibody 1 is administered at a dose of 400 mg to 800 mg, and wherein Antibody 1 comprises two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and wherein Antibody 2 comprises two heavy chains and two light chains, wherein the heavy chain comprises a HCVR having the amino acid sequence of SEQ ID NO: 5, and the light chain comprises a LCVR having the amino acid sequence of SEQ ID NO: 6.

More particularly, Antibody 1 comprises two heavy chains and two light chains, comprising the heavy chain having the amino acid sequence of SEQ ID NO: 3, and the light chain having the amino acid sequence of SEQ ID NO: 4, and wherein Antibody 2 comprises two heavy chains and two light chains, comprising the heavy chain having the amino acid sequence of SEQ ID NO: 7, and the light chain having the amino acid sequence of SEQ ID NO: 8.

More particularly, Antibody 1 is administered at a dose of 400 mg. More particularly, Antibody 1 is administered at a dose of 600 mg. More particularly, Antibody 1 is administered at a dose of 800 mg.

More particularly, Antibody 1 is administered at a dose of 400 mg on days 1 and 8 of each 3-week cycle and Antibody 2 is administered at a dose of 200 mg on day 1 of each 3-week cycle. More particularly, Antibody 1 is administered at a dose of 600 mg on days 1 and 8 of each 3-week cycle and Antibody 2 is administered at a dose of 200 mg on day 1 of each 3-week cycle. More particularly, Antibody 1 is administered at a dose of 800 mg on days 1 and 8 of each 3-week cycle and Antibody 2 is administered at a dose of 200 mg on day 1 of each 3-week cycle.

In a preferred aspect, Antibody 1 is administered by intravenous infusion. In a preferred aspect, Antibody 2 is administered by intravenous infusion.

More particularly, the non-small cell lung cancer is nonsquamous. More particularly, the non-small cell lung cancer is squamous.

The present invention provides for a kit comprising Antibody 1 and Antibody 2 for use in the treatment of non-small cell lung cancer; and wherein Antibody 1 comprises two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and wherein Antibody 2 comprises two heavy chains and two light chains, wherein the heavy chain comprises a HCVR having the amino acid sequence of SEQ ID NO: 5, and the light chain comprises a LCVR having the amino acid sequence of SEQ ID NO: 6, and wherein the amount of Antibody 1 is 400 mg to 800 mg, and the amount of Antibody 2 is 200 mg.

More particularly, Antibody 1 comprises two heavy chains and two light chains, comprising the heavy chain having the amino acid sequence of SEQ ID NO: 3, and the light chain having the amino acid sequence of SEQ ID NO: 4, and wherein Antibody 2 comprises two heavy chains and two light chains, comprising the heavy chain having the amino acid sequence of SEQ ID NO: 7, and the light chain having the amino acid sequence of SEQ ID NO: 8.

More particularly, the amount of Antibody 1 is 400 mg. More particularly, the amount of Antibody 1 is 600 mg. More particularly, the amount of Antibody 1 is 800 mg.

In a preferred aspect, Antibody 1 is administered by intravenous infusion. In a preferred aspect, Antibody 2 is administered by intravenous infusion.

More particularly, the non-small cell lung cancer is nonsquamous. More particularly, the non-small cell lung cancer is squamous.

The present invention provides for a kit comprising a first container containing a pharmaceutical composition, comprising Antibody 1 with one or more pharmaceutically acceptable carriers, diluents, or excipients, and a second container containing a pharmaceutical composition, comprising Antibody 2 with one or more pharmaceutically acceptable carriers, diluents, or excipients for use in the treatment of non-small cell lung cancer, and wherein Antibody 1 comprises two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and wherein Antibody 2 comprises two heavy chains and two light chains, wherein the heavy chain comprises a HCVR having the amino acid sequence of SEQ ID NO: 5, and the light chain comprises a LCVR having the amino acid sequence of SEQ ID NO: 6, and wherein the amount of Antibody 1 is 400 mg to 800 mg, and the amount of Antibody 2 is 200 mg.

More particularly, Antibody 1 comprises two heavy chains and two light chains, comprising the heavy chain having the amino acid sequence of SEQ ID NO: 3, and the light chain having the amino acid sequence of SEQ ID NO: 4, and wherein Antibody 2 comprises two heavy chains and two light chains, comprising the heavy chain having the amino acid sequence of SEQ ID NO: 7, and the light chain having the amino acid sequence of SEQ ID NO: 8.

More particularly, the amount of Antibody 1 is 400 mg. More particularly, the amount of Antibody 1 is 600 mg. More particularly, the amount of Antibody 1 is 800 mg.

More particularly, the non-small cell lung cancer is nonsquamous. More particularly, the non-small cell lung cancer is squamous.

The present invention also provides for a kit comprising a first container containing a pharmaceutical composition comprising Antibody 1, wherein Antibody 1 comprises two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and wherein Antibody 1 is formulated at a final concentration of 16 mg/mL with 10 mM citrate, 40 mM sodium chloride, 133 mM glycine, 50 mM mannitol, 0.01% polysorbate-80, pH6.0, and a second container containing a pharmaceutical composition comprising Antibody 2, wherein Antibody 2 comprises two heavy chains and two light chains, wherein the heavy chain comprises a HCVR having the amino acid sequence of SEQ ID NO: 5, and the light chain comprises a LCVR having the amino acid sequence of SEQ ID NO: 6, wherein Antibody 2 is formulated at a final concentration of 25 mg/mL with 9.99 mM L-histidine, 0.15 mM polysorbate 80, and 204.68 mM sucrose.

More particularly, the amount of Antibody 1 is 400 mg to 800 mg, and the amount of Antibody 2 is 200 mg. More particularly, the amount of Antibody 1 is 400 mg. More particularly, the amount of Antibody 1 is 600 mg. More particularly, the amount of Antibody 1 is 800 mg.

In a preferred aspect, Antibody 1 is administered by intravenous infusion. In a preferred aspect, Antibody 2 is administered by intravenous infusion.

More particularly, the non-small cell lung cancer is nonsquamous. More particularly, the non-small cell lung cancer is squamous.

Accordingly, in some aspects, the present invention provides a method of treating non-small cell lung cancer in a patient, comprising administering to the patient in need of such a treatment an effective amount of Antibody 1 in combination with an effective amount of Antibody 2, wherein Antibody 1 comprises two heavy chains and two light chains, wherein the heavy chain comprises a heavy chain variable region (HCVR) having the amino acid sequence of SEQ ID NO: 1, and wherein the light chain comprises a light chain variable region (LCVR) having the amino acid sequence of SEQ ID NO: 2, and wherein Antibody 2 comprises two heavy chains and two light chains, wherein the heavy chain comprises a HCVR having the amino acid sequence of SEQ ID NO: 5, and wherein the light chain comprises a LCVR having the amino acid sequence of SEQ ID NO: 6, and wherein Antibody 1 is administered at a dose of 400 mg to 800 mg, and wherein Antibody 2 is administered at a dose of 200 mg.

More particularly, Antibody 1 comprises two heavy chains and two light chains, comprising the heavy chain having the amino acid sequence of SEQ ID NO: 3, and the light chain having the amino acid sequence of SEQ ID NO: 4, and wherein Antibody 2 comprises two heavy chains and two light chains, comprising the heavy chain having the amino acid sequence of SEQ ID NO: 7, and the light chain having the amino acid sequence of SEQ ID NO: 8.

More particularly, the amount of Antibody 1 is 400 mg. More particularly, the amount of Antibody 1 is 600 mg. More particularly, the amount of Antibody 1 is 800 mg. More particularly, the amount of Antibody 2 is 200 mg.

More particularly, Antibody 1 is administered at a dose of 400 mg on days 1 and 8 of each 3-week cycle. More particularly, Antibody 1 is administered at a dose of 600 mg on days 1 and 8 of each 3-week cycle. More particularly, Antibody 1 is administered at a dose of 800 mg on days 1 and 8 of each 3-week cycle. Yet, more particularly, Antibody 2 is administered at a dose of 200 mg on day 1 of each 3-week cycle.

In a preferred aspect, Antibody 1 is administered by intravenous infusion. In a preferred aspect, Antibody 2 is administered by intravenous infusion.

More particularly, the non-small cell lung cancer is nonsquamous. More particularly, the non-small cell lung cancer is squamous.

Preferably, the combination of Antibody 1 and Antibody 2 is administered simultaneously, separately, or sequentially.

The present invention also provides for a method of treating non-small cell lung cancer in a patient, comprising administering to the patient in need of such a treatment an effective amount of Antibody 1 in combination with an effective amount of Antibody 2, wherein Antibody 1 comprises two heavy chains and two light chains, wherein the heavy chain comprises the amino acid sequence of SEQ ID NO: 3, and wherein the light chain comprises the amino acid sequence of SEQ ID NO: 4, and wherein Antibody 2 comprises two heavy chains and two light chains, wherein the heavy chain comprises the amino acid sequence of SEQ ID NO: 7, and wherein the light chain comprises the amino acid sequence of SEQ ID NO: 8, and wherein Antibody 1 is administered at a dose of 400 mg to 800 mg, and wherein Antibody 2 is administered at a dose of 200 mg.

More particularly, the amount of Antibody 1 is 400 mg. More particularly, the amount of Antibody 1 is 600 mg. More particularly, the amount of Antibody 1 is 800 mg.

More particularly, Antibody 1 is administered at a dose of 400 mg on days 1 and 8 of each 3-week cycle. More particularly, Antibody 1 is administered at a dose of 600 mg on days 1 and 8 of each 3-week cycle. More particularly, Antibody 1 is administered at a dose of 800 mg on days 1 and 8 of each 3-week cycle. Yet, more particularly, Antibody 2 is administered at a dose of 200 mg on day 1 of each 3-week cycle.

In a preferred aspect, Antibody 1 is administered by intravenous infusion. In a preferred aspect, Antibody 2 is administered by intravenous infusion.

More particularly, the non-small cell lung cancer is nonsquamous. More particularly, the non-small cell lung cancer is squamous.

Preferably, the combination of Antibody 1 and Antibody 2 is administered simultaneously, separately, or sequentially.

More particularly, wherein a tumor tissue from the patient has a protein expression level of PD-L1, and wherein the expression of PD-L1 is less than 50% of the viable tumor cells showing partial or complete membrane staining.

More particularly, a method of treating non-small cell lung cancer in a patient, comprising the steps: (1) measuring the protein expression level of PD-L1 in a tumor tissue sample taken from the patient, and (2) administering to the patient in need of such a treatment an effective amount of Antibody 1 in combination with an effective amount of Antibody 2, if the PD-L1 expression is less than 50% of the viable tumor cells showing partial or complete membrane staining.

The present invention also provides for a method of treating non-small cell lung cancer in a patient, comprising administering a 800 mg dose of Antibody 1 comprising two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and a 200 mg dose of Antibody 2 comprising two heavy chains and two light chains, wherein the heavy chain comprises a HCVR having the amino acid sequence of SEQ ID NO: 5, and the light chain comprises a LCVR having the amino acid sequence of SEQ ID NO: 6, to the patient provided that a sample of the patient's tumor tissue contains a protein expression level of PD-L1 which is less than 50% of the viable tumor cells showing partial or complete membrane staining.

The present invention also provides for necitumumab for use in simultaneous, separate, or sequential combination with pembrolizumab in the treatment of non-small cell lung cancer in a patient, wherein necitumumab is administered at a dose of 800 mg on days 1 and 8 of each 3-week cycle, and wherein pembrolizumab is administered at a dose of 200 mg on day 1 of each 3-week cycle.

The present invention also provides for pembrolizumab for use in simultaneous, separate, or sequential combination with necitumumab for the treatment of non-small cell lung cancer in a patient, wherein pembrolizumab is administered at a dose of 200 mg on day 1 of each 3-week cycle, and wherein necitumumab is administered at a dose of 800 mg on days 1 and 8 of each 3-week cycle.

The present invention also provides for use of necitumumab in the manufacture of a medicament for the treatment of non-small cell lung cancer, wherein necitumumab is administered at a dose of 800 mg in simultaneous, separate, or sequential combination with pembrolizumab, wherein pembrolizumab is administered at a dose of 200 mg.

The present invention also provides for use of pembrolizumab in the manufacture of a medicament for the treatment of non-small cell lung cancer, wherein pembrolizumab is administered at a dose of 200 mg in simultaneous, separate, or sequential combination with necitumumab administered at a dose of 800 mg.

The present invention also provides for a kit comprising necitumumab and pembrolizumab for use in the treatment of non-small cell lung cancer, wherein the amount of necitumumab is 800 mg, and the amount of pembrolizumab is 200 mg.

The present invention also provides for a kit comprising a first container containing a pharmaceutical composition, comprising necitumumab with one or more pharmaceutically acceptable carriers, diluents, or excipients, and a second container containing a pharmaceutical composition, comprising pembrolizumab with one or more pharmaceutically acceptable carriers, diluents, or excipients for use in the treatment of non-small cell lung cancer, and wherein the amount of necitumumab is 800 mg, and the amount of pembrolizumab is 200 mg.

The present invention also provides for a kit comprising a first container containing a pharmaceutical composition comprising necitumumab, and wherein necitumumab is formulated at a final concentration of 16 mg/mL with 10 mM citrate, 40 mM sodium chloride, 133 mM glycine, 50 mM mannitol, 0.01% polysorbate-80, pH6.0, and a second container containing a pharmaceutical composition comprising pembrolizumab, wherein pembrolizumab is formulated at a final concentration of 25 mg/mL with 9.99 mM L-histidine, 0.15 mM polysorbate 80, and 204.68 mM sucrose. More particularly, the amount of necitumumab is 800 mg, and wherein pembrolizumab is 200 mg.

The present invention also provides for a method of treating non-small cell lung cancer in a patient, comprising administering to the patient in need of such a treatment an effective amount of necitumumab in combination with an effective amount of pembrolizumab, wherein necitumumab is administered at a dose of 800 mg, and wherein pembrolizumab is administered at a dose of 200 mg.

The present invention also provides for a method of treating non-small cell lung cancer in a patient, comprising administering to the patient in need of such a treatment an effective amount of necitumumab in combination with an effective amount of pembrolizumab, wherein necitumumab is administered at a dose of 800 mg on days 1 and 8 of each 3-week cycle, and wherein pembrolizumab is administered at a dose of 200 mg on day 1 of each 3-week cycle.

The present invention also provides for necitumumab for use in simultaneous, separate, or sequential combination with pembrolizumab in the treatment of non-small cell lung cancer in a patient, comprising in vitro assaying a sample of the patient's tumor tissue, for the protein expression level of PD-L1, and administering a therapeutically effective amount of necitumumab and pembrolizumab, comprising administering a 800 mg dose of necitumumab, and a 200 mg dose of pembrolizumab, if the expression of PD-L1 is less than 50% of the viable tumor cells showing partial or complete membrane staining.

The present invention also provides for pembrolizumab for use in simultaneous, separate, or sequential combination with necitumumab in the treatment of non-small cell lung cancer in a patient, comprising in vitro assaying a sample of the patient's tumor tissue, for the protein expression level of PD-L1, and administering a therapeutically effective amount of pembrolizumab and necitumumab, comprising administering a 800 mg dose of necitumumab, and a 200 mg dose of pembrolizumab, if the expression of PD-L1 is less than 50% of the viable tumor cells showing partial or complete membrane staining.

The present invention also provides for a method of treating non-small cell lung cancer in a patient, comprising the steps: (1) measuring the protein expression level of PD-L1 in a tumor tissue sample taken from the patient, and (2) administering to the patient in need of such a treatment an effective amount of necitumumab in combination with an effective amount of pembrolizumab, comprising administering a 800 mg dose of necitumumab, and a 200 mg dose of pembrolizumab, if the PD-L1 expression is less than 50% of the viable tumor cells showing partial or complete membrane staining.

The present invention also provides for a method of treating non-small cell lung cancer in a patient, comprising administering a 800 mg dose of necitumumab and a 200 mg dose of pembrolizumab, to the patient provided that a sample of the patient's tumor tissue contains a protein expression level of PD-L1 which is less than 50% of the viable tumor cells showing partial or complete membrane staining.

An antibody or a full-length antibody is an immunoglobulin molecule comprising two heavy chains and two light chains interconnected by disulfide bonds. The amino terminal portion of each chain includes a variable region of about 100-110 amino acids primarily responsible for antigen recognition via the complementarity determining regions (CDRs) contained therein. The carboxy-terminal portion of each chain defines a constant region primarily responsible for effector function.

As used herein, the term “Antibody 1” refers to any antibody that comprises the following amino acid sequences:

Heavy chain variable region
(SEQ ID NO: 1)
QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSS
Light chain variable region
(SEQ ID NO: 2)
EIVMTQSPATLSLSPGERATLSCRASQSVSSYLAWYQQKPGQAPRLLIYD
ASNRATGIPARFSGSGSGTDFTLTISSLEPEDFAVYYCHQYGSTPLTFGG
GTKAEIK

An antibody with the following heavy and light chain sequences would meet the definition of Antibody 1 used herein:

| Heavy chain | |
|---|---|
| (SEQ ID NO: 3) | |
| QVQLQESGPG LVKPSQTLSL TCTVSGGSIS SGDYYWSWIR QPPGKGLEWI | 50 |
| GYIYYSGSTD YNPSLKSRVT MSVDTSKNQF SLKVNSVTAA DTAVYYCARV | 100 |
| SIFGVGTFDY WGQGTLVTVS SASTKGPSVL PLAPSSKSTS GGTAALGCLV | 150 |
| KDYFPEPVTV SWNSGALTSG VHTFPAVLQS SGLYSLSSVV TVPSSSLGTQ | 200 |
| TYICNVNHKP SNTKVDKRVE PKSCDKTHTC PPCPAPELLG GPSVFLFPPK | 250 |
| PKDTLMISRT PEVTCVVVDV SHEDPEVKFN WYVDGVEVHN AKTKPREEQY | 300 |
| NSTYRVVSVL TVLHQDWLNG KEYKCKVSNK ALPAPIEKTI SKAKGQPREP | 350 |
| QVYTLPPSRE EMTKNQVSLT CLVKGFYPSD IAVEWESNGQ PENNYKTTPP | 400 |
| VLDSDGSFFL YSKLTVDKSR WQQGNVFSCS VMHEALHNHY TQKSLSLSPG | 450 |
| K | 451 |
| Light chain | |
| (SEQ ID NO: 4) | |
| EIVMTQSPAT LSLSPGERAT LSCRASQSVS SYLAWYQQKP GQAPRLLIYD | 50 |
| ASNRATGIPA RFSGSGSGTD FTLTISSLEP EDFAVYYCHQ YGSTPLTFGG | 100 |
| GTKAEIKRTV AAPSVFIFPP SDEQLKSGTA SVVCLLNNFY PREAKVQWKV | 150 |
| DNALQSGNSQ ESVTEQDSKD STYSLSSTLT LSKADYEKHK VYACEVTHQG | 200 |
| LSSPVTKSFN RGEC | 214 |

In one aspect of the invention, “necitumumab” refers to any antibody that is necitumumab as defined by the International Nonproprietary Name (INN) found in WHO Drug Information Vol 23, No. 3, 2009, pp. 253-254. As mentioned above, necitumumab is the active pharmaceutical ingredient in Portrazza®. It has also been identified as IMC-11F8, and CAS registry number 906805-06-9. Necitumumab is one example of Antibody 1.

As used herein, the term “Antibody 2” refers to any antibody that comprises the following amino acid sequences:

Heavy chain variable region
(SEQ ID NO: 5)
QVQLVQSGVEVKKPGASVKVSCKASGYTFTNYYMYWVRQAPGQGLEWMGGI
NPSNGGTNFNEKFKNRVTLTTDSSTTTAYMELKSLQFDDTAVYYCARRDYR
FDMGFDYWGQGTTVTVSS
Light chain variable region
(SEQ ID NO: 6)
EIVLTQSPATLSLSPGERATLSCRASKGVSTSGYSYLHWYQQKPGQAPRLL
IYLASYLESGVPARFSGSGSGTDFTLTISSLEPEDFAVYYCQHSRDLPLTF
GGGTKVEIK

An antibody with the following heavy and light chain sequences would meet the definition of Antibody 2 used herein:

| Heavy chain | |
|---|---|
| (SEQ ID NO: 7) | |
| QVQLVQSGVE VKKPGASVKV SCKASGYTFT NYYMYWVRQA PGQGLEWMGG | 50 |
| INPSNGGTNF NEKFKNRVTL TTDSSTTTAY MELKSLQFDD TAVYYCARRD | 100 |
| YRFDMGFDYW GQGTTVTVSS ASTKGPSVFP LAPCSRSTSE STAALGCLVK | 150 |
| DYFPEPVTVS WNSGALTSGV HTFPAVLQSS GLYSLSSVVT VPSSSLGTKT | 200 |
| YTCNVDHKPS NTKVDKRVES KYGPPCPPCP APEFLGGPSV FLFPPKPKDT | 250 |
| LMISRTPEVT CVVVDVSQED PEVQFNWYVD GVEVHNAKTK PREEQFNSTY | 300 |
| RVVSVLTVLH QDWLNGKEYK CKVSNKGLPS SIEKTISKAK GQPREPQVYT | 350 |
| LPPSQEEMTK NQVSLTCLVK GFYPSDIAVE WESNGQPENN YKTTPPVLDS | 400 |
| DGSFFLYSRL TVDKSRWQEG NVFSCSVMHE ALHNHYTQKS LSLSLGK | 447 |
| Light chain | |
| (SEQ ID NO: 8) | |
| EIVLTQSPAT LSLSPGERAT LSCRASKGVS TSGYSYLHWY QQKPGQAPRL | 50 |
| LIYLASYLES GVPARFSGSG SGTDFTLTIS SLEPEDFAVY YCQHSRDLPL | 100 |
| TFGGGTKVEI KRTVAAPSVF IFPPSDEQLK SGTASVVCLL NNFYPREAKV | 150 |
| QWKVDNALQS GNSQESVTEQ DSKDSTYSLS STLTLSKADY EKHKVYACEV | 200 |
| THQGLSSPVT KSFNRGEC | 218 |

In one aspect of the invention, “pembrolizumab” refers to any antibody that is pembrolizumab as defined by the International Nonproprietary Name (INN) found in WHO Drug Information Vol. 28, No. 3, 2014, p. 407. As mentioned above, pembrolizumab is the active pharmaceutical ingredient in Keytruda®. It has also been identified as MK-3475; SCH-900475; lambrolizumab; lambrolizumab, and CAS registry number 1374853-91-4. Pembrolizumab is one example of Antibody 2.

As used herein, the terms “treating,” “to treat,” or “treatment” refers to restraining, slowing, stopping, reducing, or reversing the progression or severity of an existing symptom, disorder, condition, disease, or cancer.

As used herein, the term “patient” refers to a mammal, preferably a human.

As used herein, the terms “cancer” and “cancerous” refers to or describes the physiological condition in patients that is typically characterized by unregulated cell growth. Included in this definition are benign and malignant cancers. Examples of cancer include, NSCLC among others.

As used herein, the term “kit” refers to a package comprising at least two separate containers, wherein a first container contains a pharmaceutical composition comprising Antibody 1 and a second container contains a pharmaceutical composition comprising Antibody 2. A “kit” may also include instructions to administer all or a portion of the contents of these first and second containers to a cancer patient.

A potential advantage of the combination treatments of the invention is the possibility of producing marked and/or prolonged anti-cancer effects in a patient with an acceptable safety profile including acceptable tolerability, toxicities and/or adverse events, so that the patient benefits from the combination treatment method overall. The efficacy of the combination treatment of the invention can be measured by various endpoints commonly used in evaluating cancer treatments, including but not limited to, tumor regression, tumor weight or size shrinkage, time to progression, overall survival, progression free survival, overall response rate, duration of response, and quality of life. The therapeutic agents used in the invention may cause inhibition of metastatic spread without shrinkage of the primary tumor, or may simply exert a tumoristatic effect. Because the invention relates to the use of a unique combination of anti-tumor agents, various approaches to determining efficacy of any particular combination therapy of the present invention can be optionally employed, including, for example, cell-cycle dependent biomarkers measurement/visualization, and measurement of response through radiological imaging.

As used herein, the term Complete Response (CR) is defined as the disappearance of all target lesions. Any pathological lymph nodes (whether target or non-target) must have reduction in short axis to <10 mm. Tumor marker results must have normalized.

As used herein, the term Partial Response (PR) is defined as at least a 30% decrease in the sum of diameter of target lesions, taking as reference the baseline sum diameters.

As used herein the term Progressive Disease (PD) is defined as at least a 20% increase in the sum of the diameters of target lesions, taking as reference the smallest sum on study (including the baseline sum if that is the smallest). In addition to the relative increase of 20%, the sum must also demonstrate an absolute increase of at least 5 mm. The appearance of 1 or more new lesions is also considered progression. For equivocal findings of progression (for example, very small and uncertain new lesions; cystic changes or necrosis in existing lesions), treatment may continue until the next scheduled assessment. If at the next scheduled assessment, progression is confirmed, the date of progression should be the earlier date when progression was suspected.

As used herein, the term Stable Disease (SD) is defined as neither sufficient shrinkage to qualify for PR nor sufficient increase to qualify for PD, taking as reference the smallest sum diameters while on study.

As used herein, the term Not Evaluable (NE) is define as when an incomplete radiologic assessment of target lesions is performed or there is a change in the method of measurement from baseline that impacts the ability to make a reliable evaluation of response.

As used herein, the term Progression-Free Survival (PFS) is defined as the time from the date of first dose of any study drug until the date of radiographically documented PD or death due to any cause, whichever is earlier.

As used herein, the term Overall Survival (OS) is defined as the time from the date of study first dose of any study drug to the date of death from any cause.

As used herein, the term Objective Response Rate is defined as the proportion of patients achieving a best overall response of PR or CR.

As used herein, the term Disease Control Rate (DCR) is defined as the proportion of patients achieving a best overall response of SD, PR, or CR.

As used herein, the term Overall Response Rate is based on each patient's best objective response and will be determined for all patients evaluable via the Response Evaluation Criteria in Solid Tumors (RECIST) v 1.1 criteria. The Overall Response Rate (%) will be calculated as the number of patients with best objective response of CR or PR divided by the number of patients with measurable disease at baseline. The best objective response for a given patient will be based on objective responses determined from data obtained up to: progression or the last evaluable assessment in the absence of progression. Patients for whom an objective response cannot be determined, or for who the best objective response is NE, will be considered non-responders. The Overall Response Rate will be summarized along with the 95% Clopper Pearson confidence interval.

As used herein, the term “effective amount” refers to the amount or dose of Antibody 1, including but not limited to necitumumab, and/or to the amount or dose of Antibody 2, including but not limited to pembrolizumab, which, upon single or multiple dose administration to the patient, provides an effective response in the patient under diagnosis or treatment. It is also understood that a combination therapy of the present invention is carried out by administering Antibody 1, including but not limited to necitumumab, together with Antibody 2, including but not limited to pembrolizumab, in any manner which provides effective levels of Antibody 1, including but not limited to necitumumab, and Antibody 2, including but not limited to pembrolizumab, in the body.

An effective amount can be readily determined by one skilled in the art, by the use of known techniques, and by observing results obtained under analogous circumstances. In determining the effective amount for a patient, a number of factors are considered by the attending diagnostician, including, but not limited to: the species of patient; its size, age, and general health; the specific disease or disorder involved; the degree of, or involvement, or the severity of the disease or disorder; the response of the individual patient; the particular compound administered; the mode of administration; the bioavailability characteristics of the preparation administered; the dose regimen selected; the use of concomitant medication; and other relevant circumstances.

As used herein, the term “effective response” of a patient or a patient's “responsiveness” to treatment with a combination of agents and similar wording refers to the clinical or therapeutic benefit imparted to a patient upon co-administration of Antibody 2, including but not limited to pembrolizumab, and Antibody 1, including but not limited to necitumumab. Such benefit includes any one or more of: extending survival (including OS and PFS); resulting in an objective response (including a CR or a PR); or improving signs or symptoms of cancer, etc.

Antibody 1, including but not limited to necitumumab, is generally effective over a dosage range in the combination of the present invention. For example, dosages per 21-day cycle normally fall within the range of about 400 to 800 mg with dosages on 2 days of that cycle, preferably about 400 to 800 mg on Day 1 and Day 8 of each 21-day cycle, more preferably about 600 to 800 mg on Day 1 and Day 8 of each 21-day cycle, and most preferably about 800 mg on Day 1 and Day 8 of each 21-day cycle. Antibody 2, including but not limited to pembrolizumab, is generally effective over a dosage range in the combination of the present invention. For example, dosages per 21-day cycle normally fall within the range of about 1 mg/kg to about 3 mg/kg as an intravenous infusion over 30 minutes, more preferably about 2 mg/kg as an intravenous infusion over 30 minutes. Alternatively, dosages per 21-day cycle normally fall within the range of about 100 mg to about 300 mg as an intravenous infusion over 30 minutes, more preferably about 200 mg as an intravenous infusion over 30 minutes.

Dosage regimens may be adjusted for patient safety or to provide the optimum desired response (e.g., a therapeutic response). Dosing schedules, for intravenous (i.v.) or non-intravenous administration, localized or systemic, or combinations thereof, will typically range from a single bolus dosage or continuous infusion, to multiple administrations per day (e.g., every 4-6 hours), or as indicated by the treating physician and the patient's condition. As used herein, intravenous infusion and intravenous injection can be used interchangeably.

As used herein, the phrase “in combination with” refers to the administration of Antibody 1 with Antibody 2 simultaneously. As used herein, the phrase “in combination with” also refers to the administration of Antibody 1 with Antibody 2 sequentially in any order. As used herein, the phrase “in combination with” also refers to the administration of Antibody 1 with Antibody 2 in any combination thereof. Antibody 2 can be administered prior to administration of Antibody 1. Antibody 2 can be administered at the same time as administration of Antibody 1. Antibody 2 can be administered subsequent to administration of Antibody 1. Antibody 2 can be administered prior to, at the same time as, or subsequent to administration of Antibody 1, or in some combination thereof.

Where Antibody 1 is administered at repeated intervals (e.g. during a standard course of treatment), Antibody 2 can be administered prior to each administration of Antibody 1. Where Antibody 1 is administered at repeated intervals (e.g. during a standard course of treatment), Antibody 2 can be administered at the same time as each administration of Antibody 1. Where Antibody 1 is administered at repeated intervals (e.g. during a standard course of treatment), Antibody 2 can be administered subsequent to each administration of Antibody 1. Where Antibody 1 is administered at repeated intervals (e.g. during a standard course of treatment), Antibody 2 can be administered prior to, at the same time as, or subsequent to, each administration of Antibody 1 or some combination thereof. Where Antibody 1 is administered at repeated intervals (e.g. during a standard course of treatment), Antibody 2 can be administered at different intervals in relation to therapy with Antibody 1. Where Antibody 1 is administered at repeated intervals (e.g. during a standard course of treatment), Antibody 2 can be administered in a single or series of dose(s) prior to, at any time during, or subsequent to the course of treatment with Antibody 1. Where Antibody 1 is administered at repeated intervals (e.g. during a standard course of treatment), Antibody 2 can be administered in a single dose prior to, at any time during, or subsequent to the course of treatment with Antibody 1. Where Antibody 1 is administered at repeated intervals (e.g. during a standard course of treatment), Antibody 2 can be administered in a single dose prior to the course of treatment with Antibody 1. Where Antibody 1 is administered at repeated intervals (e.g. during a standard course of treatment), Antibody 2 can be administered in a single dose at any time during the course of treatment with Antibody 1. Where Antibody 1 is administered at repeated intervals (e.g. during a standard course of treatment), Antibody 2 can be administered in a single dose subsequent to the course of treatment with Antibody 1. Where Antibody 1 is administered at repeated intervals (e.g. during a standard course of treatment), Antibody 2 can be administered in a series of doses prior to the course of treatment with Antibody 1. Where Antibody 1 is administered at repeated intervals (e.g. during a standard course of treatment), Antibody 2 can be administered in a series of doses subsequent to the course of treatment with Antibody 1. Where Antibody 1 is administered at repeated intervals (e.g. during a standard course of treatment), Antibody 2 can be administered in a series of doses subsequent to the course of treatment with Antibody 1.

The invention contemplates using a PD-1 ligand, more particularly PD-L1, as a biomarker for cancer patients who respond to treatment when the cancer has PD-1 expression. The invention further contemplates the method of predicting successful treatment of a patient with Antibody 1 in combination Antibody 2 by measuring the PD-L1 levels in tumor tissue of the patient.

One aspect of the invention is a method of treating non-small cell lung cancer in a patient, comprising the steps: (1) measuring the level of PD-L1 in a tumor tissue sample taken from the patient, and (2) administering to the patient in need of such a treatment an effective amount of Antibody 1 in combination with an effective amount of Antibody 2, if the PD-L1 levels are less than 50% of the viable tumor cells showing partial or complete membrane staining.

The present invention also contemplates a method for determining whether a subject having a cancer is a candidate for Antibody 1 in combination Antibody 2 comprising: (1) ex vivo or in vitro determining the protein expression level of PD-L1 in a tumor tissue sample of the subject; and (2) wherein the PD-L1 levels are less than 50% of the viable tumor cells showing partial or complete membrane staining, is indicative that the subject is a candidate for the combination.

The present invention also contemplates a therapeutic regimen for treating non-small cell lung cancer comprising: a) selecting a patient having non-small cell lung cancer and whose protein expression level of PD-L1 is less than 50% of the viable tumor cells showing partial or complete membrane staining, and b) administering to the patient a 800 mg dose of Antibody 1 comprising two heavy chains and two light chains, wherein the heavy chain comprises a HCVR having the amino acid sequence of SEQ ID NO: 1, and wherein the light chain comprises a LCVR having the amino acid sequence of SEQ ID NO: 2, on days 1 and 8 of each 3-week cycle, and a 200 mg dose of Antibody 2 comprising two heavy chains and two light chains, wherein the heavy chain comprises a HCVR having the amino acid sequence of SEQ ID NO: 5, and the light chain comprises a LCVR having the amino acid sequence of SEQ ID NO: 6, on day 1 of each 3-week cycle.

The present invention contemplates a method of treating non-small cell lung cancer in a patient, comprising administering a 800 mg dose of Antibody 1 comprising two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and a 200 mg dose of Antibody 2 comprising two heavy chains and two light chains, wherein the heavy chain comprises a HCVR having the amino acid sequence of SEQ ID NO: 5, and the light chain comprises a LCVR having the amino acid sequence of SEQ ID NO: 6, to the patient provided that a sample of the patient's tumor tissue contains a protein expression level of PD-L1 which is less than 50% of the viable tumor cells showing partial or complete membrane staining.

The present invention also contemplates an in vitro method of selecting a patient having non-small cell lung cancer for treatment with a 800 mg dose of Antibody 1 comprising two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and a 200 mg dose of Antibody 2 comprising two heavy chains and two light chains, wherein the heavy chain comprises a HCVR having the amino acid sequence of SEQ ID NO: 5, and the light chain comprises a LCVR having the amino acid sequence of SEQ ID NO: 6, comprising testing for the protein expression level of PD-L1 in a tumor tissue sample from the patient, wherein the patient is selected if the protein expression level of PD-L1 is less than 50% of the viable tumor cells showing partial or complete membrane staining.

The present invention also contemplates a method of identifying patients with non-small cell lung cancer eligible for treatment with a 800 mg dose of Antibody 1 comprising two heavy chains and two light chains, wherein the heavy chain comprises a HCVR having the amino acid sequence of SEQ ID NO: 1, and wherein the light chain comprises a LCVR having the amino acid sequence of SEQ ID NO: 2, and a 200 mg dose of Antibody 2 comprising two heavy chains and two light chains, wherein the heavy chain comprises a HCVR having the amino acid sequence of SEQ ID NO: 5, and the light chain comprises a LCVR having the amino acid sequence of SEQ ID NO: 6, comprising testing a tumor tissue sample from the patient for the protein expression level of PD-L1, wherein the patient is eligible for treatment with said combination of Antibody 1 and Antibody 2 if the protein expression level of PD-L1 is less than 50% of the viable tumor cells showing partial or complete membrane staining.

PD-L1 expression levels can be measured in a variety of methods including qualitative immunohistochemical assay, such as the companion diagnostic approved by the FDA for KEYTRUDA® (pembrolizumab), PD-L1 IHC 22C3 pharmDx. IHC PD-L1 IHC 22C3 pharmDx is indicated as an aid in identifying NSCLC patients for treatment with KEYTRUDA® (pembrolizumab). “PD-L1 IHC 22C3 pharmDx is a qualitative immunohistochemical assay using Monoclonal Mouse Anti-PD-L1, Clone 22C3 intended for use in the detection of PD-L1 protein in formalin-fixed, paraffin-embedded (FFPE) non-small cell lung cancer (NSCLC) tissue using EnVision FLEX visualization system on Autostainer Link 48. PD-L1 protein expression is determined by using Tumor Proportion Score (TPS), which is the percentage of viable tumor cells showing partial or complete membrane staining.” (IHC PD-L1 IHC 22C3 pharmDx, Approval Order Letter for PMA P150013, 2 Nov. 2015.)

The following examples and clinical study results further illustrate the present invention.

The following clinical study designs further illustrate the invention, but should not be construed to limit the scope of the invention in any way.

Necitumumab or Antibody 1 can be made, for example, according to the disclosure in WO 2005/090407. Pembrolizumab or Antibody 2 can be made, for example, according to the disclosure in WO 2008/156712.

### A Study of Necitumumab in Combination with Pembrolizumab for Patients with Stage IV NSCLC

Clinical Study Design

Open label, single-arm, multicenter Phase 1b study (hereinafter “Study”) to investigate the effectiveness, safety, and tolerability of necitumumab combined with pembrolizumab in ˜65 patients with Stage IV NSCLC including squamous and nonsquamous NSCLC. See NCT02451930 available on clinicaltrials.gov. The Study consists of several parts including:
Part A: escalating doses of necitumumab (600 mg and 800 mg IV) were administered on Day 1 and 8 every 3 weeks (Q3W) in combination with pembrolizumab (200 mg IV) on Day 1 Q3W in patients with Stage IV NSCLC.
Part B: expansion cohort with the dose of necitumumab identified in Part A in combination with a fixed regimen of pembrolizumab (200 mg IV) on Day 1 Q3W in patients with Stage IV NSCLC.

Approximately 54 patients will be enrolled in Part B and split approximately evenly between patients with squamous and nonsquamous histology.

Patients will be treated until PD, toxicity requiring cessation, protocol noncompliance, or withdrawal of consent. Patients who are on study therapy at study completion may continue to receive study therapy in the continued access period until they meet the discontinuation criteria. Study completion is expected approximately 6-12 months after the last patient has been enrolled.

Major eligibility criteria include: progression after 1 platinum-based chemotherapy; ECOG PS 0-1; no prior treatment with anti-PD-1, -PD-L1, or -PD-L2 agents; and no symptomatic brain metastases.

PD-L1 and EGFR status will be assessed in archived tissue samples. PD-L1 expression levels will be measured using the companion diagnostic approved by the FDA for KEYTRUDA® (pembrolizumab), PD-L1 IHC 22C3 pharmDx.

A PD-L1 negative status is assigned to a patient when PD-L1 protein expression is less than 1% of the viable tumor cells showing partial or complete membrane staining as determined by using Tumor Proportion Score (TPS). A PD-L1 low positive status is assigned to a patient when PD-L1 protein expression is between 1-49% of the viable tumor cells showing partial or complete membrane staining as determined by using Tumor Proportion Score (TPS). A PD-L1 high positive status is assigned to a patient when PD-L1 protein expression is greater than 50% of the viable tumor cells showing partial or complete membrane staining as determined by using Tumor Proportion Score (TPS).

Study Objectives

The primary objective of Part A of the Study is to investigate safety and tolerability of pembrolizumab 200 mg Q3W when combined with necitumumab administered at the doses of 600 mg and 800 mg on Days 1 and 8 of 21-day cycles in patients with Stage IV NSCLC (all histologies) as measured by number of patients with a DLT during Cycle 1; and for Part B to evaluate the efficacy of necitumumab in combination with pembrolizumab in terms of ORR by RECIST 1.1 in patients with Stage IV NSCLC of squamous and nonsquamous histology.

The secondary objectives of the Study include: for Part A: (i) to investigate the safety profile as assessed by significant clinical and laboratory events of necitumumab in combination with pembrolizumab; (ii) to determine the ORR (by RECIST 1.1); (iii) to determine PK of necitumumab in presence of pembrolizumab; (iv) to determine the immunogenicity of necitumumab in presence of pembrolizumab; for Part B: (v) to demonstrate the feasibility of combining necitumumab with pembrolizumab at the recommended doses by: (1) investigating the safety profile as assessed by clinical and laboratory significant events, and determining disease control rate (DCR), duration of response (DOR), and PFS by RECIST 1.1, and OS; (2) determining PK of necitumumab in the presence of pembrolizumab; and (3) determining the immunogenicity of necitumumab in the presence of pembrolizumab. The correlation of potential biomarkers to clinical outcome may also be assessed.

Trial Drugs:

Necitumumab is a sterile, preservative-free, I.V. infusion supplied at a final concentration of 16 mg/mL (800 mg/50 mL) contained in single-use vials, in a formulation of 10 mM citrate, 40 mM sodium chloride, 133 mM glycine, 50 mM mannitol, 0.01% polysorbate-(TWEEN®)-80, pH 6.0.

Pembrolizumab is a sterile, preservative-free, white to off-white lyophilized powder in single-use vials. Each vial is reconstituted and diluted for intravenous infusion. Each 2 mL of reconstituted solution contains 50 mg of pembrolizumab and is formulated in L-histidine (3.1 mg), polysorbate 80 (0.4 mg), and sucrose (140 mg). May contain hydrochloric acid/sodium hydroxide to adjust pH to 5.5.

Study Results:

Part A completed without dose-limiting toxicity (9 patients; 2 squamous, 7 nonsquamous). Part B (expansion study) commenced in December 2015 using necitumumab 800 mg.

The clinical characteristics of the patients were typical of those with advanced non-small-cell lung cancer; notably there were slightly more than expected patients no PD-L1 expression (50% with no expression). Of note, 7/34 (21%) patients were never smokers, and 3/34 (9%) patients were treated with EGFR TKI as first-line therapy, suggesting presence of EGFR mutation. Historically, these patients responded poorly to PD-1 directed antibodies.

Based on the six month interim efficacy analysis, being also the primary outcome analysis for the nonsquamous NSCLC cohort, the data for all lesions (confirmed and unconfirmed) from 34 patients (7 nonsquamous patients from part A and 27 nonsquamous patients from Part B) illustrates an objective response rate of 29% and a disease control rate of 68% for patients receiving the combination of necitumumab and pembrolizumab. The median PFS is 6.9 months, (with a 95% confidence interval [CI] 2.7-NR), and the six-month PFS rate is 55% (95% CI 36-71). Best overall responses showed: 0 CR, 10 PR (29%), 12 SD (38%), 7 PD (21%) and 4 Not evaluated due to no post-baseline scan (12%).

Accordingly, the 6 month interim results illustrate that necitumumab in combination with pembrolizumab provides an improved ORR and PFS over historical single agent activity of pembrolizumab seen in Keynote 001 study (KN001) (Garon E et al, N Engl J Med 2015; 372:2018-28). When compared to the median PFS and 6-month PFS rate of pembrolizumab alone as seen in the KN001 Study, the combination of necitumumab and pembrolizumab illustrates a numerical benefit as compared to pembrolizumab based on historical data.

Based on PD-L1 status, the overall response rate was 18.0% in the 17 PD-L1-negative patients, 60% in the 5 PD-L1 low positive patients and 40% in the 5 PD-L1 high-positive patients, as compared to 9%, 15% and 41% for the same category in KN001, respectively. When patients who never smoked and those who received a previous EGFR TKI were excluded, the ORR was 38%, which included a response rate of 25% in PD-L1-non expressing patients, 75% in the PD-L1-low expressing patients and 50% in PD-L1 high expressing patients.

Accordingly, a PD-L1 expression below 50% indicates an improved efficacy response to the combination of necitumumab and pembrolizumab as compared to pembrolizumab.

The safety profile of the combination corresponds to individual profiles for both drugs, with no additive toxicities.

### Feb. 11, 2016 Data Cut-Off

18 pts (neci 600 mg n=3, 800 mg n=15) were eligible for inclusion. Patients were female 44.4%, had median age 66.5 years [range 48-76], and adenocarcinoma histology 77.8%. All pts experienced ≥1 treatment-emergent adverse event (AE) with ≥1 related to study treatment (Table 1). Four serious AEs occurred in 3 (16.7%) pts (all respiratory and mediastinal); none were treatment-related. No discontinuations or deaths were attributable to AEs. AEs occurring with >15% frequency are listed in the table below. Four (22.2%) pts experienced 8 grade >2 AEs: acute respiratory failure, hypokalaemia, hypophosphataemia, infusionrelated reaction, pulmonary embolism, raised gamma-glutamyl transferase (1 pt each), and dyspnoea (2 pts).

TABLE 1

| | Interim Safety Population | |
|---|---|---|
| MedDRA Preferred Term | (N = 18) | |
| Dermatitis acneiform | 16 | (88.9) |
| Dry skin | 8 | (44.4) |
| Asthenia | 7 | (38.9) |
| Appetite decreased | 4 | (22.2) |
| Constipation | 4 | (22.2) |
| Headache | 4. | (2.22) |
| Hypoalbuminaemia | 4 | (22.2) |
| Hypophosphataemia | 4 | (22.2) |
| Pruritus | 4 | (22.2) |
| Anaemia | 3 | (16.7) |
| Diarrhoea | 3 | (16.7) |
| Dyspnoea | 3 | (16.7) |
| Fatigue | 3 | (16.7) |
| Hypokalaemia | 3 | (16.7) |
| Respiratory tract infection | 3 | (16.7) |
| Stomatitis | 3 | (16.7) |

The combination of necitumumab and pembrolizumab appears tolerable. The safety profile corresponds to individual profiles for both drugs, with no additive toxicities.

### Additional Clinical Disclosure

This phase 1b, multicenter, single arm study of necitumumab and pembrolizumab examined the safety, efficacy, and tolerability in pretreated patients with Stage IV NSCLC (NCT02451930). PDL1 was centrally assessed retrospectively using IHC 22C3 pharmDx assay (negative, weak positive, strong positive if <1%, 1-49%, ≥50% of tumor cells were stained, respectively). Escalating doses of necitumumab 600-800 mg IV (days 1 and 8 every 3 weeks [Q3W]) were administered with fixed dose of pembrolizumab (200 mg IV) on Day 1 Q3W (Part A). Established dose of necitumumab from Part A was used in the expansion cohort (Part B). Study objectives for part A were to determine the dose-limiting toxicity (DLT) and evaluate tolerability and overall response rate (ORR) by RECIST 1.1.

Part A (dose escalation of necitumumab in combination with standard dose of pembrolizumab) of this study completed without dose-limiting toxicities (DLTs); the combination was tolerable. No additive, new or unexpected adverse events (AEs) were seen and the safety results corresponded to those of each drug

The Part B primary objective is to evaluate the ORR by RECIST 1.1 of necitumumab when combined with pembrolizumab in patients with Stage IV NSCLC who have progressed after 1 platinum-based chemotherapy regimen.

Part B secondary objectives are to determine disease control rate (DCR), duration of response (DoR), and progression-free survival (PFS) by RECIST 1.1, safety, and OS; immunogenicity, and pharmacokinetics (PK) of necitumumab in the presence of pembrolizumab.

A total of 64 pateints of squamous (n=30) and non-squamous (n=34) histology were enrolled (Parts A and B). Results for 64 patients are reported. Patient demographis and characteristics are shown in Table 2. Efficacy results by PDL1 status and histology are also shown in Table 2. PDL1 was centrally assessed retrospectively using IHC 22C3 pharmDx assay (negative, weak positive, strong positive if <1%, 1-49%, ≥50% of tumor cells were stained, respectively).

Tumor response was assessed radiographically according to RECIST 1.1. Initial tumor imaging was performed within 21 days of the first dose of treatment; subsequent image assessments were performed every 6 weeks thereafter.

The null hypothesis is based on the assumption that the ORR is 20% and the alternative response rate of the combination treatment on ORR is 35%. Sample size of 54 evaluable patients (27 squamous and 27 nonsquamous) in Part B provides statistical power of 83%, with a nominal one-sided alpha level of 0.10.

Baseline characteristic and efficacy results overall and by histology are provided in Table 2.

The results suggest modest activity of the combination in a NSCLC patient population with a relatively high proportion of PDL1 negative tumors.

In all evaluable patients, regardless of histology or PDL1 status: ORR (confirmed) 23.4% (95% CI, 13.8%-35.7%); DCR (confirmed) 64.1% (95% CI, 51.1%-75.7%); mPFS 4.1 mo (95% CI, 2.4-6.9); and OS rate @ 6 months 74.7% (95% CI, 61.5%-83.9%).

ORR was similar in both squamous and non-squamous histology (20.0% vs 26.5%, respectively). However, PFS was better in the nonsquamous than squamous cohort (mPFS 6.9 mo vs 2.8 mo, respectively).

Efficacy appeared related to the PDL1 status of strong positive, weak positive, and negative, respectively: ORR 40.0%, 25.0%, and 12.5%; and mPFS 7.6 mo, 5.4 mo and 2.7 mo.

TABLE 2

| | Necitumumab 600 mg/800 mg + Pembrolizumab 200 mg | | | | | |
|---|---|---|---|---|---|---|
| | Overall | | Squamous | | Nonsquamous | |
| | (N = 64) | | (n = 30) | | (n = 34) | |
| Age, median (range), y | 65 | (43, 81) | 67.5 | (48, 81) | 61 | (43, 75) |
| Male, n (%) | 46 | (71.9) | 23 | (76.7) | 23 | (67.6) |

Prior systemic therapy, n (%)

| 1 line | 36 | (56.3) | 22 | (73.3) | 14 | (41.2) |
|---|---|---|---|---|---|---|
| 2 lines | 15 | (23.4) | 5 | (16.7) | 10 | (29.4) |
| ≥3 lines | 13 | (20.3) | 3 | (10.0) | 10 | (29.4) |
| Baseline ECOG PS, n (%) | 64 | (100) | 30 | (100) | 34 | (100) |
| 0 | 17 | (26.6) | 3 | (10.0) | 14 | (41.2) |
| 1 | 46 | (71.9) | 26 | (86.7) | 20 | (58.8) |
| 2 | 1 | (1.6) | 1 | (3.3) | 0 | |
| Tobacco use, n (%) | 64 | (100) | 30 | (100) | 34 | (100) |
| Former | 41 | (64.1) | 21 | (70.0) | 20 | (58.8) |
| Current | 14 | (21.9) | 7 | (23.3) | 7 | (20.6) |
| Never | 9 | (14.1) | 2 | (6.7) | 7 | (20.6) |

Efficacy

| ORR n (%) (95% CI) | 15 | (23.4) | 6 | (20.0) | 9 | (26.5) |
|---|---|---|---|---|---|---|
| | (13.8, 35.7) | | (7.7, 38.6) | | (12.9, 44.4) | |
| mPFS (months) | 4.1 | (2.4, 6.9) | 2.8 | (1.4, 5.5) | 6.9 | (1.5, 12.3) |
| (95% CI) | | | | | | |
| 6-month OS rate (%) | 74.7 | (61.5, 83.9) | 63.6 | (42.8, 78.6) | 84.2 | (66.0, 93.1) |
| (95% CI) | | | | | | |
| PDL1 Status | 64 | (100) | 30 | (100) | 34 | (100) |
| PDL1 negative | 32 | (50.0) | 13 | (43.3) | 19 | (55.9) |
| ORR n (%) (95% CI) | 4 | (12.5) | 1 | (7.7) | 3 | (15.8) |
| | (3.5, 29.0) | | (0.2, 36.0) | | (3.4, 39.6) | |
| mPFS (m) (95% CI) | 2.69 | (1.4, 4.1) | | | | |
| 6-month OS rate (%) | 68.2 | (47.7, 82.0) | | | | |
| (95% CI) | | | | | | |
| PDL1 Weak positive | 12 | (18.8) | 7 | (23.3) | 5 | (14.7) |
| ORR n (%) (95% CI) | 3 | (25.0) | 1 | (14.3) | 2 | (40.0) |
| | (5.5, 57.2) | | (0.4, 57.9) | | (5.3, 85.3) | |
| mPFS (m) (95% CI) | 5.4 | (0.8, NR) | | | | |
| 6-month OS rate (%) | 83.3 | (48.2, 95.6) | | | | |
| (95% CI) | | | | | | |
| PDL1 Strong positive | 10 | (15.6) | 5 | (16.7) | 5 | (14.7) |
| ORR n (%) (95% CI) | 4 | (40.0) | 2 | (40.0) | 2 | (40.0) |
| | (12.2, 73.8) | | (5.3, 85.3) | | (5.3, 85.3) | |
| mPFS (m) (95% CI) | 7.6 | (1.0, 12.3) | | | | |
| 6-month OS rate (%) | 80.0 | (40.9, 94.6) | | | | |
| (95% CI) | | | | | | |
| Unknown | 10 | (15.6) | 5 | (16.7) | 5 | (14.7) |
| ORR n (%) (95% CI) | 4 | (40.0) | 2 | (40.0) | 2 | (40.0) |
| | (12.2, 73.8) | | (5.3, 85.3) | | (5.3, 85.3) | |
| mPFS (m) (95% CI) | NR | (0.82, NR) | | | | |
| 6-month OS rate (%) | 78.8 | (38.1, 94.3) | | | | |
| (95% CI) | | | | | | |

ECOG PS, Eastern Cooperative Oncology Group performance status;
PDL1, programmed death ligand 1;
ORR, overall response rate;
mPFS, median progression-free survival;
OS, overall survival;
CI, confidence interval;
NR, not reached;
m, month(s).

The safety profile of the combination corresponded to individual profiles of both therapy components, and no additive effect was seen, Treatment-emergent adverse events in ≥10% of pateints are shown in Table 3. An overview of safety is shown in Table 4. Although the majority of patients experienced treatment-related AEs (TRAE) of any grade, less than 10% of patients experienced at least 1 grade 3 or higher TRAE.

TABLE 3

| | Total (N = 64) | | | |
|---|---|---|---|---|
| | Number of Patients (%) | | | |
| MedDRA Preferred Term | Any Grade | | Grade ≥ 3 | |
| Dermatitis acneiform | 43 | (67) | 3 | (5) |
| Asthenia | 24 | (38) | 3 | (5) |
| Dry skin | 23 | (36) | 0 | |
| Hypomagnesemia | 21 | (33) | 4 | (6) |
| Fatigue | 20 | (31) | 4 | (6) |
| Decreased Appetite | 17 | (27) | 0 | |
| Diarrhoea | 17 | (27) | 1 | (2) |
| Headache | 14 | (22) | 0 | |
| Pruritis | 14 | (22) | 1 | (2) |
| Stomatitis | 14 | (22) | 1 | (2) |
| Arthralgia | 12 | (19) | 1 | (2) |
| Cough | 11 | (17) | 0 | |
| Dyspnea | 11 | (17) | 6 | (9) |
| Constipation | 10 | (16) | 0 | |
| Skinn fissures | 10 | (16) | 0 | |
| Paronychia | 8 | (13) | 0 | |
| Hirsuitism^{a} | 2 | (11) | 0 | |
| Alanine aminotransferase | 7 | (11) | 1 | (2) |
| increased | | | | |
| Anemia | 7 | (11) | 0 | |
| Hypokalemia | 7 | (11) | 3 | (5) |
| Hypophosphatemia | 7 | (11) | 2 | (3) |

| | | | |
|---|---|---|---|
| Nausea | 7 | (11) | 0 |
| Skin infection | 7 | (11) | 0 |
| Vomiting | 7 | (11) | 0 |
| Xerosis | 7 | (11) | 0 |

^{a}Denominator adjusted for female patients only

TABLE 4

| | Total (N = 64) | |
|---|---|---|
| | Number of Patients | |
| | (%) | |
| Any treatment-emergent adverse events (TEAEs) | 64 | (100) |
| Grade ≥ 3 TEAEs | 40 | (63) |
| Treatement-related adverse events (TRAEs) | 60 | (94) |
| Grade ≥ 3 TRAEs | 20 | (31) |
| Serious TEAEs | 27 | (42) |
| Serious TRAEs | 11 | (17) |
| TEAEs leading to discontinuation of study treatment | 6 | (9) |
| AEs with outcome of death^{b} | 2 | (3) |

^{b}Two Grade 5 respiratory tract infections

The results suggest activity of the necitumumab and pembrolizumab combination in a pretreated NSCLC patient population with a relatively high proportion of PDL1 negative patients. The safety profile corresponded to individual profiles for both drugs, with no additive toxicities. The combination of PD 1 and EGFR blockade may be a therapeutic strategy to extend the duration of treatment response and delay development of resistance in the biomarker-selected population.

Sequences
<SEQ ID NO: 1; PRT1; Artificial sequence>
Heavy chain variable region of necitumumab
QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSS
<SEQ ID NO: 2; PRT1; Artificial sequence>
Light chain variable region of necitumumab
EIVMTQSPATLSLSPGERATLSCRASQSVSSYLAWYQQKPGQAPRLLIYD
ASNRATGIPARFSGSGSGTDFTLTISSLEPEDFAVYYCHQYGSTPLTFGG
GTKAEIK
<SEQ ID NO: 3; PRT1; Artificial sequence>
Heavy chain of necitumumab
QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSSASTKGPSVLPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K
<SEQ ID NO: 4; PRT1; Artificial sequence>
Light chain of necitumumab
EIVMTQSPATLSLSPGERATLSCRASQSVSSYLAWYQQKPGQAPRLLIYD
ASNRATGIPARFSGSGSGTDFTLTISSLEPEDFAVYYCHQYGSTPLTFGG
GTKAEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
<SEQ ID NO: 5; PRT1; Artificial sequence>
Heavy chain variable region of pembrolizumab
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS
<SEQ ID NO: 6; PRT1; Artificial sequence>
Light chain variable region of pembrolizumab
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIK
<SEQ ID NO: 7; PRT1; Artificial sequence>
Heavy chain of pembrolizumab
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALISGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
<SEQ ID NO: 8; PRT1; Artificial sequence>
Light chain of pembrolizumab
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

1.-21. (canceled)

22. A kit comprising Antibody 1 and Antibody 2, wherein Antibody 1 comprises two heavy chains and two light chains, wherein the Antibody 1 heavy chains comprise a HCVR having the amino acid sequence of SEQ ID NO: 1, and wherein the Antibody 1 light chains comprise a LCVR having the amino acid sequence of SEQ ID NO: 2, and wherein Antibody 2 comprises two heavy chains and two light chains, wherein the Antibody 2 heavy chains comprise a HCVR having the amino acid sequence of SEQ ID NO: 5, and the Antibody 2 light chains comprise a LCVR having the amino acid sequence of SEQ ID NO: 6, and wherein the amount of Antibody 1 is 400 mg to 800 mg, and the amount of Antibody 2 is 200 mg.

23. The kit of claim 22, wherein the amount of Antibody 1 is 800 mg.

24. (canceled)

25. (canceled)

26. The kit of claim 22, comprising a first container containing a pharmaceutical composition comprising Antibody 1 with one or more pharmaceutically acceptable carriers, diluents, or excipients, and a second container containing a pharmaceutical composition comprising Antibody 2 with one or more pharmaceutically acceptable carriers, diluents, or excipients.

27.-29. (canceled)

30. The kit of claim 26, wherein Antibody 1 is formulated at a final concentration of 16 mg/mL with 10 mM citrate, 40 mM sodium chloride, 133 mM glycine, 50 mM mannitol, 0.01% polysorbate-80, pH6.0, and Antibody 2 is formulated at a final concentration of 25 mg/mL with 9.99 mM L-histidine, 0.15 mM polysorbate 80, and 204.68 mM sucrose.

31. (canceled)

32. (canceled)

33. A method of treating non-small cell lung cancer in a patient, comprising administering to the patient in need of such a treatment an effective amount of Antibody 1 in combination with an effective amount of Antibody 2, wherein Antibody 1 comprises two heavy chains and two light chains, wherein the Antibody 1 heavy chains comprise a heavy chain variable region (HCVR) having the amino acid sequence of SEQ ID NO: 1, and wherein the Antibody 1 light chains comprise a light chain variable region (LCVR) having the amino acid sequence of SEQ ID NO: 2, and wherein Antibody 2 comprises two heavy chains and two light chains, wherein the Antibody 2 heavy chains comprise a HCVR having the amino acid sequence of SEQ ID NO: 5, and the Antibody 2 light chains comprise a LCVR having the amino acid sequence of SEQ ID NO: 6, and wherein Antibody 1 is administered at a dose of 400 mg to 800 mg, and wherein Antibody 2 is administered at a dose of 200 mg.

34. The method of claim 33, wherein Antibody 1 comprises two heavy chains and two light chains, wherein the Antibody 1 heavy chains comprise the amino acid sequence of SEQ ID NO: 3, and wherein the Antibody 1 light chains comprise the amino acid sequence of SEQ ID NO: 4, and wherein Antibody 2 comprises two heavy chains and two light chains, wherein the Antibody 2 heavy chains comprise the amino acid sequence of SEQ ID NO: 7, and the Antibody 2 light chains comprise the amino acid sequence of SEQ ID NO: 8.

35. The method of claim 33, wherein the amount of Antibody 1 is 800 mg.

36. The method of claim 33, wherein Antibody 1 is administered at a dose of 800 mg on days 1 and 8 of each 3-week cycle.

37. The method of claim 33, wherein Antibody 2 is administered at a dose of 200 mg on day 1 of each 3-week cycle.

38. The method of claim 33, wherein the combination of Antibody 1 and Antibody 2 is administered simultaneously, separately, or sequentially.

39. The method of claim 33, wherein the non-small cell lung cancer is nonsquamous.

40. The method of claim 33, wherein the non-small cell lung cancer is squamous.

41. The method of claim 33, wherein a tumor tissue from the patient has a the protein expression level of PD-L1, and wherein the expression of PD-L1 is less than 50% of the viable tumor cells showing partial or complete membrane staining.

42.-45. (canceled)

46. The kit of claim 26, further comprising instructions to administer all or a portion of the contents of the first and second containers to a cancer patient.

47. The kit of claim 46, wherein the cancer is non-small cell lung cancer.

48. The kit of claim 47, wherein the non-small cell lung cancer is nonsquamous.

49. The kit of claim 47, wherein the non-small cell lung cancer is squamous.
